(19) United States
(12) Patent Application Publication
(10) Pub. No.: US 2008/0091227 A1
(43) Pub. Date: Apr. 17, 2008

(54) SURGICAL PROBE AND METHOD OF MAKING

(75) Inventors: Gregory SCHMITZ, Los Gatos, CA (US); Ron Leguidleguid, Fremont, CA (US)

Correspondence Address: SHAY GLENN LLP, 2755 CAMPUS DRIVE, SUITE 210, SAN MATEO, CA, 94403, US

(73) Assignee: Baxano, Inc., Mountain View, CA (US)

(21) Appl. No.: 11/843,561

(22) Filed: Aug. 22, 2007

Related U.S. Application Data
(60) Provisional application No. 60/823,594, filed on Aug. 25, 2006.

(51) Int. Cl. A61B 17/00

(52) U.S. Cl. 606/190; 29/557

(57) ABSTRACT

A method for making a probe for placing a guidewire between two tissues in a patient's body may involve forming at least one bend in a thin, rigid tube having a proximal end and a distal end, flattening at least part of the tube closer to the distal end than the proximal end, and removing a portion of the upper surface of the flattened part of the tube to form an aperture. A probe for placing a guidewire between two tissues in a patient's body may include: a thin, rigid tube having a proximal straight portion, a flattened distal portion, a bend disposed between the proximal and distal portions, and a lumen passing through the proximal portion and at least part of the distal portion; and a curved, flexible tubular member disposed at least partially within the lumen of the tube.

### CROSS-REFERENCE TO RELATED APPLICATIONS

The present application claims priority under 35 U.S.C. §119 to U.S. Provisional Patent Application 60/823,594, entitled “Surgical Probe and Method of Making” filed Aug. 25, 2006 which is incorporated herein by reference in its entirety.

### BACKGROUND OF THE INVENTION

The present invention relates generally to medical/surgical devices and methods. More specifically, the present invention relates to a surgical probe and method for making same.

In recent years, less invasive (or “minimally invasive”) surgical techniques have become increasingly more popular, as physicians, patients and medical device innovators have sought to achieve similar or improved outcomes, relative to conventional surgery, while reducing the trauma, recovery time and side effects typically associated with conventional surgery. Developing less invasive surgical methods and devices, however, can pose many challenges. For example, some challenges of less invasive techniques include working in a smaller operating field, working with smaller devices, and trying to operate with reduced or even no direct visualization of the structure (or structures) being treated. These challenges are compounded by the fact that target tissues to be modified often reside very close to one or more vital, non-target tissues, which the surgeon hopes not to damage. Two initial obstacles in any given minimally invasive procedure, therefore, are accessing a desired location within the patient and positioning a minimally invasive surgical device in the location to perform the procedure on one or more target tissues, while avoiding damage to nearby non-target tissues.

Examples of less invasive surgical procedures include laparoscopic procedures, arthroscopic procedures, and minimally invasive approaches to spinal surgery, such as a number of less invasive intervertebral disc removal, repair and replacement techniques. One area of spinal surgery in which a number of less invasive techniques have been developed is the treatment of spinal stenosis. Spinal stenosis occurs when neural and/or neurovascular tissue in the spine becomes impinged by one or more structures pressing against them, causing one or more symptoms. This impingement of tissue may occur in one or more of several different areas in the spine, such as in the central spinal canal, or more commonly in the lateral recesses of the spinal canal and/or one or more intervertebral foramina.

FIGS. 1-3 show various partial views of the lower (lumbar) region of the spine. FIG. 1 shows an approximate top view of a vertebra with the cauda equina (the bundle of nerves that extends from the base of the spinal cord through the central spinal canal) shown in cross section and two nerve roots exiting the central spinal canal and extending through intervertebral foramina on either side of the vertebra. The spinal cord and cauda equina run vertically along the spine through the central spinal canal, while nerve roots branch off of the spinal cord and cauda equina between adjacent vertebrae and extend through the intervertebral foramina. Intervertebral foramina may also be seen in FIGS. 2 and 3, and nerves extending through the foramina may be seen in FIG. 2.

One common cause of spinal stenosis is buckling and thickening of the ligamentum flavum (one of the ligaments attached to and connecting the vertebrae), as shown in FIG. 1. (Normal ligamentum flavum is shown in cross section in FIG. 3) Buckling or thickening of the ligamentum flavum may impinge on one or more neurovascular structures, dorsal root ganglia, nerve roots and/or the spinal cord itself. Another common cause of neural and neurovascular impingement in the spine is hypertrophy of one or more facet joints (or “zygopophaseal joints”), which provide articulation between adjacent vertebrae. (Two vertebral facet superior articular processes are shown in FIG. 1. Each superior articular process articulates with an inferior articular process of an adjacent vertebra to form a zygopophaseal joint. Such a joint is labeled in FIG. 3.) Other causes of spinal stenosis include formation of osteophytes (or “bone spurs”) on vertebrae, spondylolisthesis (sliding of one vertebra relative to an adjacent vertebra), facet joint synovial cysts, and collapse, bulging or herniation of an intervertebral disc into the central spinal canal. Disc, bone, ligament or other tissue may impinge on the spinal cord, the cauda equina, branching spinal nerve roots and/or blood vessels in the spine to cause loss of function, ischemia and even permanent damage of neural or neurovascular tissue. In a patient, this may manifest as pain, impaired sensation and/or loss of strength or mobility.

In the United States, spinal stenosis occurs with an incidence of between 4% and 6% of adults aged 50 and older and is the most frequent reason cited for back surgery in patients aged 60 and older. Conservative approaches to the treatment of symptoms of spinal stensosis include systemic medications and physical therapy. Epidural steroid injections may also be utilized, but they do not provide long lasting benefits. When these approaches are inadequate, current treatment for spinal stenosis is generally limited to invasive surgical procedures to remove ligament, cartilage, bone spurs, synovial cysts, cartilage, and bone to provide increased room for neural and neurovascular tissue. The standard surgical procedure for spinal stenosis treatment includes laminectomy (complete removal of the lamina (see FIGS. 1 and 2) of one or more vertebrae) or laminotomy (partial removal of the lamina), followed by removal (or “resection”) of the ligamentum flavum. In addition, the surgery often includes partial or occasionally complete facetectomy (removal of all or part of one or more facet joints). In cases where a bulging intervertebral disc contributes to neural impingement, disc material may be removed surgically in a discectomy procedure.

Removal of vertebral bone, as occurs in laminectomy and facetectomy, often leaves the effected area of the spine very unstable, leading to a need for an additional highly invasive fusion procedure that puts extra demands on the patient's vertebrae and limits the patient's ability to move. In a spinal fusion procedure, the vertebrae are attached together with some kind of support mechanism to prevent them from moving relative to one another and to allow adjacent vertebral bones to fuse together. Unfortunately, a surgical spine fusion results in a loss of ability to move the fused section of the back, diminishing the patient's range of motion and causing stress on the discs and facet joints of adjacent vertebral segments. Such stress on adjacent vertebrae often leads to further dysfunction of the spine, back pain, lower leg weakness or pain, and/or other symptoms. Furthermore, using current surgical techniques, gaining sufficient access to the spine to perform a laminectomy, facetectomy and spinal fusion requires dissecting through a wide incision on the back and typically causes extensive muscle damage, leading to significant post-operative pain and lengthy rehabilitation. Discectomy procedures require entering through an incision in the patient's abdomen and navigating through the abdominal anatomy to arrive at the spine. Thus, while laminectomy, facetectomy, discectomy, and spinal fusion frequently improve symptoms of neural and neurovascular impingement in the short term, these procedures are highly invasive, diminish spinal function, drastically disrupt normal anatomy, and increase long-term morbidity above levels seen in untreated patients. Although a number of less invasive techniques and devices for spinal stenosis surgery have been developed, these techniques still typically require removal of significant amounts of vertebral bone and, thus, typically require spinal fusion.

Therefore, it would be desirable to have a device for accessing an area in a patient's body, such as the spine, using minimally invasive techniques and/or for positioning a less invasive device in the area. Ideally, such a device would be less invasive than currently available techniques and would be relatively inexpensive to produce. At least some of these objectives will be met by the present invention.

### SUMMARY OF THE INVENTION

In one aspect of the present invention, a method for making a probe for placing a guidewire between two tissues in a patient's body may involve: forming at least one bend in a thin, rigid tube having a proximal end and a distal end, wherein the bend is formed closer to the distal end than the proximal end; flattening at least part of the tube closer to the distal end than the proximal end, wherein the flattened part comprises an upper surface facing the acute angle of the bend and a lower surface facing the oblique angle of the bend; and removing a portion of the upper surface of the flattened part of the tube to form an aperture.

In some embodiments, the aperture may extend onto the bend in the tube. Optionally, the method may further include bending the distal end of the tube to form an angled distal tip. The method may also include cutting off a distal end of the tube to form a new distal end, wherein the removed portion of the upper surface extends to the new distal end. In such embodiments, bending the distal end may involve bending the new distal end. Some embodiments may further involve forming a groove in the distal tip for guiding a curved, flexible, tubular member. Optionally, the method may further involve passing a curved, flexible, tubular member through the rigid tube. In some embodiments, the curved tubular member includes a curved portion toward a distal end, and the curved member may comprise a shape memory material capable of transitioning from a straight configuration, while passing through a straight portion of the rigid tube, to a curved portion, upon passing through the aperture of the rigid tube.

In some embodiments, the method may further include forming a handle over a proximal portion of the tube. In some embodiments, at least a distal portion of the tube may be configured to be passed into an epidural space and into an intervertebral foramen of a spine.

In another aspect of the present invention, a method for making a probe for placing a guidewire between two tissues in a patient's body may involve forming two halves of a metal probe comprising a proximal handle portion and a curved distal portion and coupling the two halves of the probe together, such that a lumen is formed through the two halves, extending from the proximal handle through at least a portion of the curved distal portion. In some embodiments, the method may also include passing a curved, flexible, tubular member through the lumen of the probe. For example, the curved tubular member may include a curved portion toward a distal end, and the curved member may comprise a shape memory material capable of transitioning from a straight configuration, while passing through the handle of the probe, to a curved portion, upon passing out of the lumen. In some embodiments, at least the curved distal portion of the probe may be configured to be passed into an epidural space and into an intervertebral foramen of a spine.

In another aspect of the present invention, a probe for placing a guidewire between two tissues in a patient's body may include a thin, rigid tube having a proximal straight portion, a flattened distal portion, a bend disposed between the proximal and distal portions, and a lumen passing through the proximal portion and at least part of the distal portion, and a curved, flexible tubular member disposed at least partially within the lumen of the tube, wherein at least a portion of the tubular member is configured to change from a straight configuration in the proximal portion of the tube to a curved configuration upon exiting the tube.

Optionally, the probe may also include an angled distal tip at the extreme distal end of the distal portion of the tube, wherein the angled distal tip is angled relative to the distal portion. In some embodiments, the lumen extends from a proximal aperture in the proximal portion of the tube to a distal aperture in the distal portion. In such embodiments, the distal aperture may optionally extend from the bend onto the distal portion. In some embodiments, the flexible tubular member may a shape memory material. In some embodiments, a lumen of the flexible ember may have an inner diameter sufficient to allow passage of a therethrough.

These and other aspects and embodiments are described more fully the Detailed Description, with reference to the attached Drawings.

### BRIEF DESCRIPTION OF THE DRAWINGS

FIG. 1 is cross-sectional view of a spine, showing a top view of a lumbar vertebra, a cross-sectional view of the cauda equina, and two exiting nerve roots;

FIG. 2 is a left lateral view of the lumbar portion of a spine with sacrum and coccyx;

FIG. 3 is a left lateral view of a portion of the lumbar spine, showing only bone and ligament tissue and partially in cross section;

FIG. 4 is a cross-sectional view of a patient's back and spine with a side views of a guidewire and tissue modification system in place for performing a tissue removal procedure, according to one embodiment of the present invention;

FIGS. 5A-5I are cross-sectional views of a patient's back and spine, with side views of several devices demonstrating a method for accessing and placing a tissue modification device in the spine, according to one embodiment of the present invention;

FIG. 6, is perspective view of a surgical probe formed from a hypotube, according to one embodiment of the present invention;

FIG. 7, is perspective view of a surgical probe formed from a hypotube and including a handle, according to an alternative embodiment of the present

FIGS. 8A-8G, are perspective and side views showing a method for making a surgical probe using a hypotube, according to one embodiment of the present invention;

FIG. 9A, is perspective view of a surgical probe, according to an alternative embodiment of the present invention;

FIGS. 9B-9E are side, front, front and cross-sectional views, respectively, of the surgical probe of FIG. 9A;

FIGS. 9F-9H are side cross-sectional views of a distal portion of the surgical probe of FIG. 9A;

FIGS. 10A and 10B are perspective and side views, respectively, of curved, flexible, tubular member for use in a surgical probe, according to an alternative embodiment of the present invention;

FIG. 11A, is perspective view of a surgical probe, according to an alternative embodiment of the present invention;

FIGS. 11B-11E are side, front, front and cross-sectional views, respectively, of the surgical probe of FIG. 11A;

FIGS. 11F-11H are side views of a distal portion of the surgical probe of FIG. 11A; and

FIG. 12 is a side view of a system for accessing and modifying tissue in a spine, according to one embodiment of the present invention.

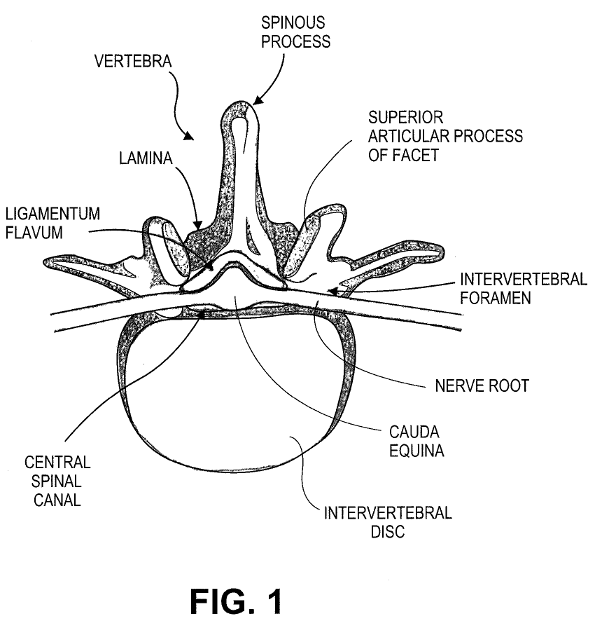

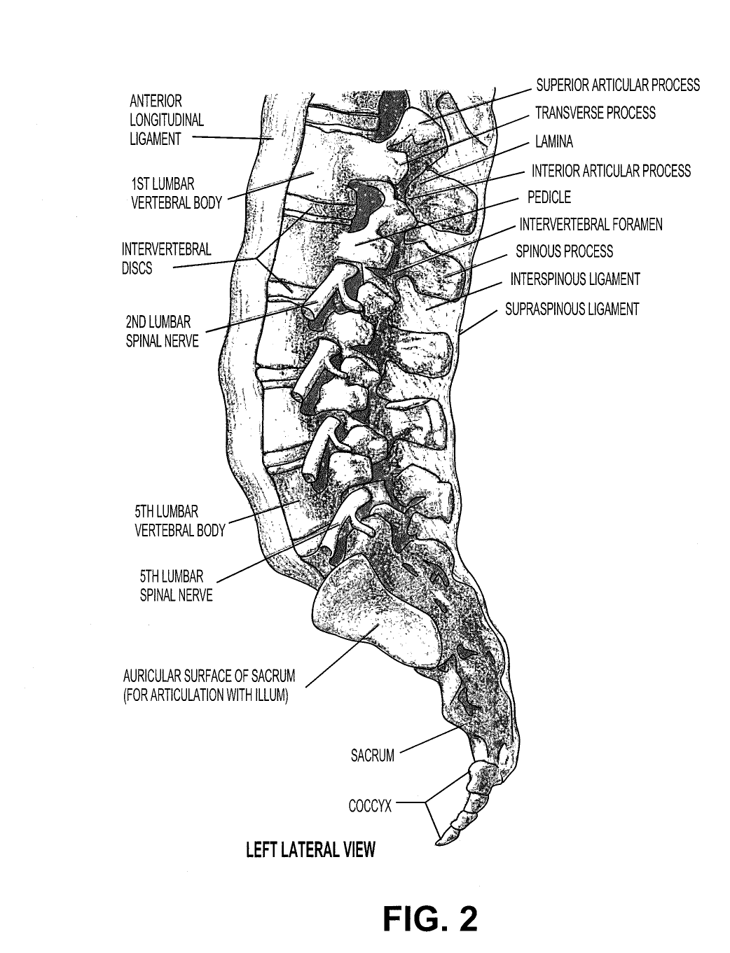

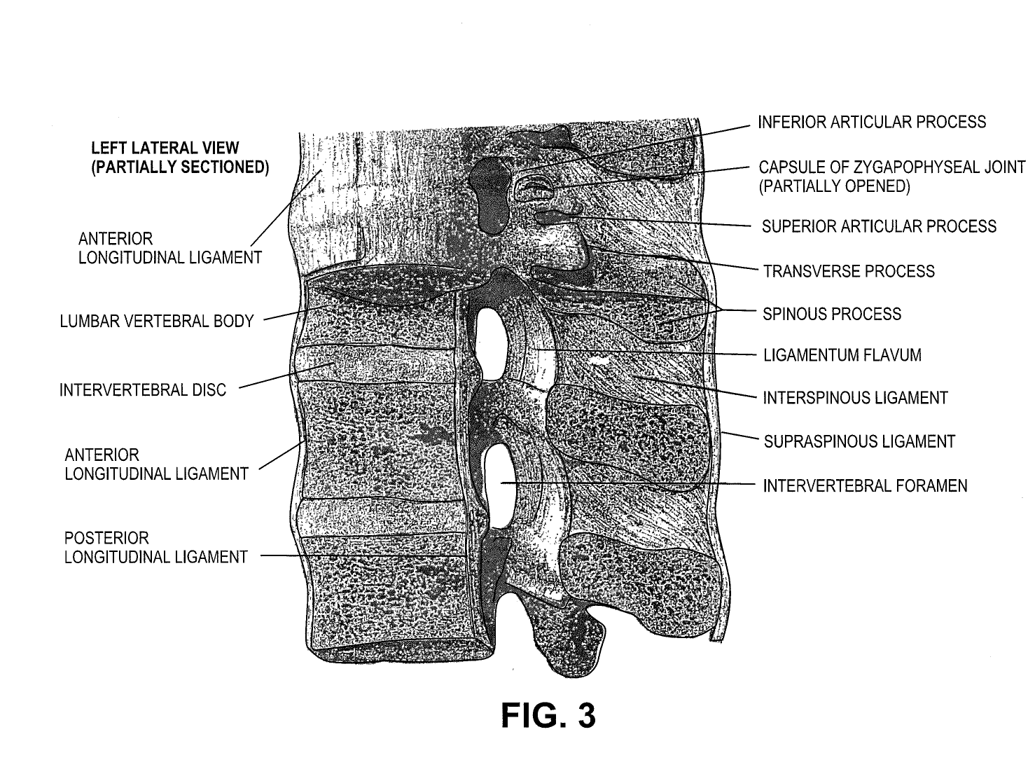

### DETAILED DESCRIPTION OF THE INVENTION

Various embodiments of a surgical probe and method for making same are provided. Although the following description and accompanying drawing figures generally focus on use of a probe in the spine, in alternative embodiments, the described probes or variations thereof may be used in any of a number of other anatomical locations in a patient's body.

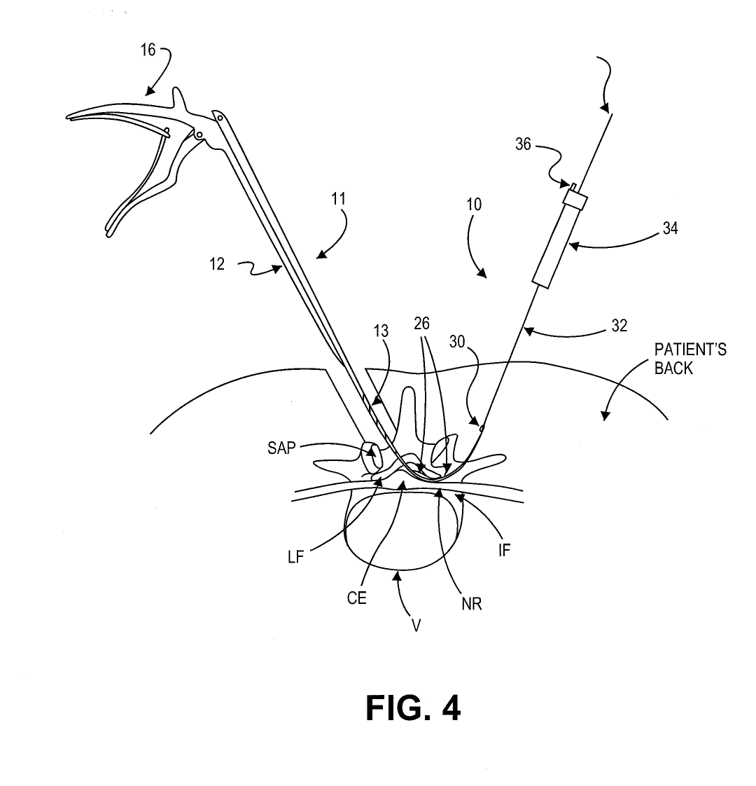

Referring to FIG. 4, one embodiment of a guidewire system 10 is shown coupled with a tissue cutting device 11 in position within a patient's spine. Further description of various embodiments of cutting device 11 may be found in U.S. patent application Ser. No. 11/461,740, entitled “Multi-Wire Tissue Cutter” (Attorney-Docket No. 026445-000900US), and filed Aug. 1, 2006, the full disclosure of which is hereby incorporated by reference. A number of alternative embodiments of cutting devices, many of which may be used (or adapted for use) with guidewire system 10, are further described in U.S. patent application Ser. Nos.: 11/375,265, entitled “Methods and Apparatus for Tissue Modification” (Original Attorney-Docket No. 78117-200101), and filed Mar. 13, 2006; 11/405,848, entitled “Mechanical Tissue Modification Devices and Methods” (Original Attorney-Docket No. 78117-200301), and filed Apr. 17, 2006; 11/406,486, entitled “Powered Tissue Modification Devices and Methods” (Original Attorney-Docket No. 78117-200501US), and filed Apr. 17, 2006; and 11/429,377, entitled “Flexible Tissue Rasp” (Original Attorney-Docket No. 78117-200201), and filed May 4, 2006. The full disclosures of all of the foregoing references are hereby incorporated by reference.

As described in further detail in U.S. patent application Ser. No. 11/461,740, tissue cutting device 11 may include a shaft 12, a proximal handle 16, a flexible distal portion 13, two or more cutting blades 26 and a guidewire coupling member 30. Guidewire system 10 may include a guidewire 32 having a sharpened tip 33 (often referred to herein as the “sharpened distal tip”) for facilitating advancement of guidewire 32 through tissue. Optionally, guidewire 32 may also include a shaped member (not visible in FIG. 4) at the end opposite sharpened tip 33 (often referred to herein as the guidewire “proximal end”) for coupling with coupling member 30. Guidewire system 10 may also include a guidewire handle 34 (or “distal handle”) for coupling with guidewire 32, which in some cases may include a tightening member 36 for securing a portion of guidewire 32 within guidewire handle 34.

In some embodiments, cutting device 11 may be advanced into a patient's back through an incision 20, which is shown in FIG. 4 as an open incision but which may be a minimally invasive or less invasive incision in alternative embodiments. In some embodiments, device 11 may be advanced by coupling guidewire connector 30 with guidewire 32 that has been advanced between target and non-target tissues, and then pulling guidewire 32 to pull device 11 between the tissues. Various embodiments of such a method for delivering a device are described in further detail below. Generally, guidewire system 10 may be used to pull flexible distal portion 13 into place between tissues in hard-to-reach or tortuous areas of the body, such as between a nerve root (NR) and facet joint and through an intervertebral foramen (IF). Generally, flexible portion 13 may be advanced to a position such that blades 26 face tissue to be cut in a tissue removal procedure (“target tissue”) and a non-cutting surface (or surfaces) of flexible portion 13 faces non-target tissue, such as nerve and/or neurovascular tissue. In the embodiment shown in FIG. 4, blades 26 are positioned to cut ligamentum flavum (LF) and may also cut hypertrophied bone of the facet joint, such as the superior articular process (SAP). (Other anatomical structures depicted in FIG. 4 include the vertebra (V) and cauda equina (CE)). In various alternative embodiments, flexible portion 13 may be replaced with a curved, rigid portion, a steerable portion, a straight portion with a distal extension or the like. The configuration, dimensions, flexibility, steerability, materials and the like of flexible portion 13 may be adjusted, in alternative embodiments, depending on a type of tissue or anatomical structure to be accessed or modified.

Before or after blades 26 are located in a desired position, guidewire 32 may be removably coupled with guidewire handle 34, such as by passing guidewire 32 through a central bore in handle 34 and moving tightening member 36 to secure a portion of guidewire 32 within handle 34. A physician (or two physicians or one physician and an assistant) may then pull on proximal handle 16 and distal handle 34 to apply tensioning force to guidewire 32 and cutting device 11 and to urge the cutting portion of device 11 against ligamentum flavum (LF), superior articular process (SAP), or other tissue to be cut. Proximal handle 16 may then be actuated, such as by squeezing in the embodiment shown, to cause one or both blades 26 to move toward one another to cut tissue. Proximal handle 16 may be released and squeezed as many times as desired to remove a desired amount of tissue. When a desired amount of tissue has been cut, guidewire 32 may be released from distal handle 34, and cutter device 11 and guidewire 32 may be removed from the patient's back.

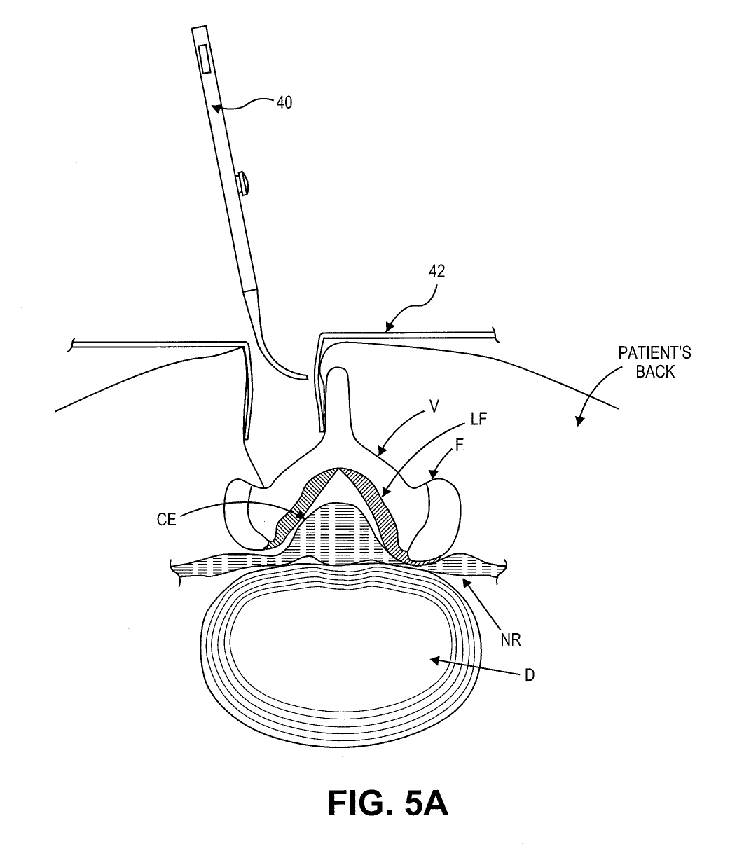

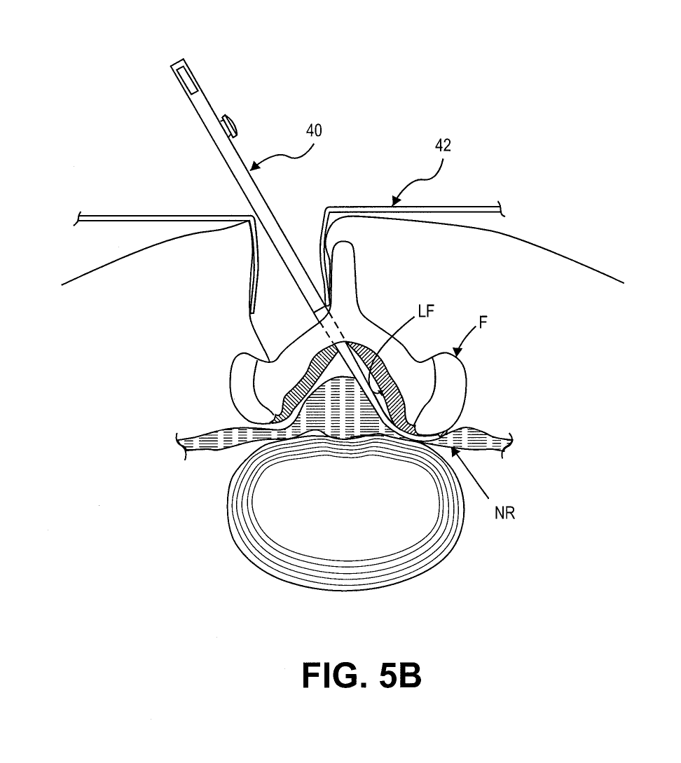

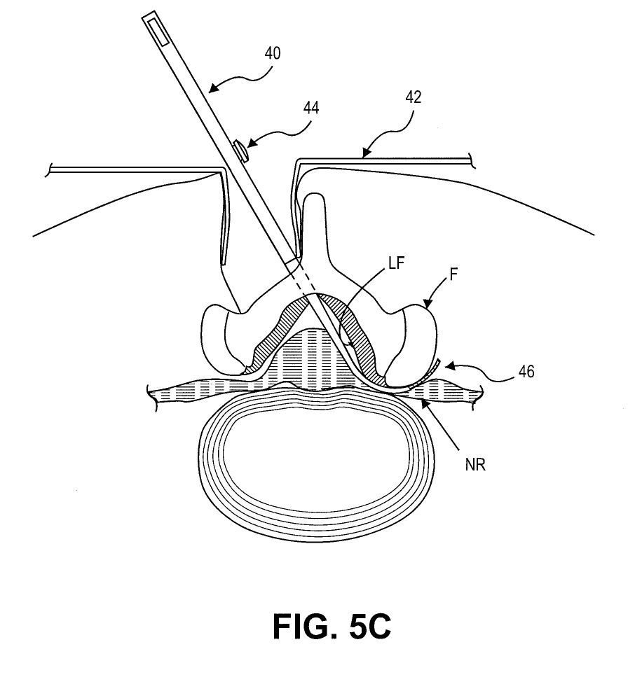

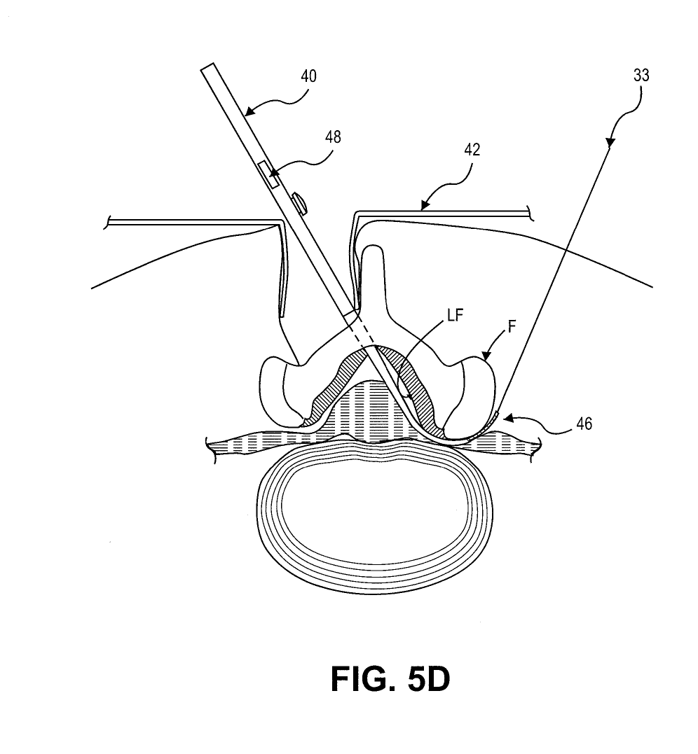

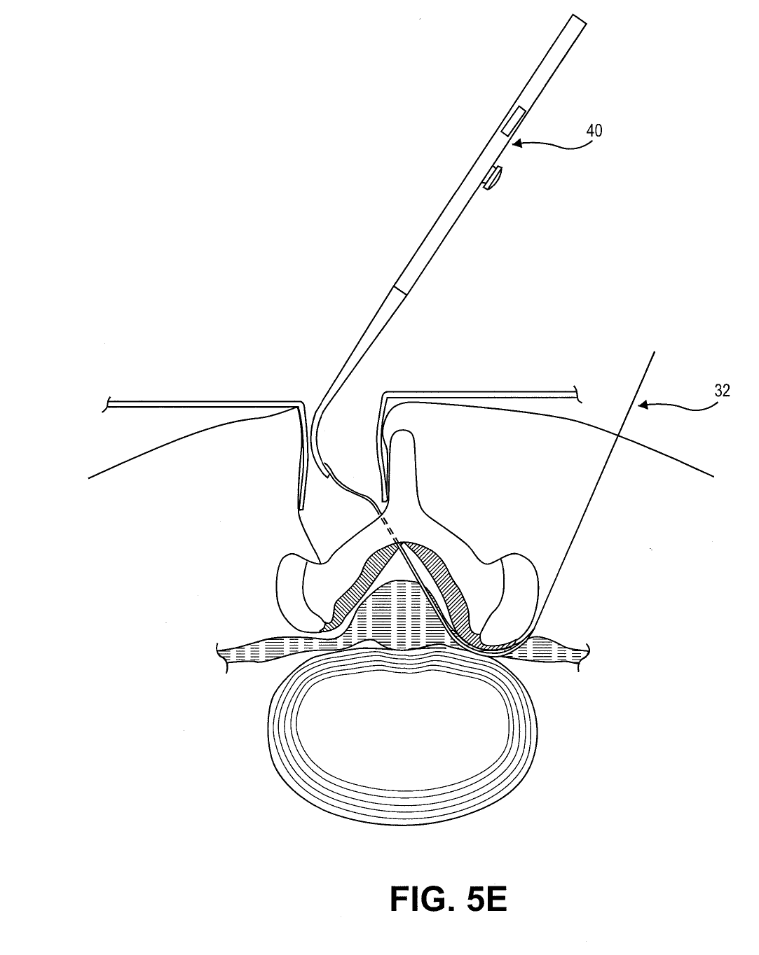

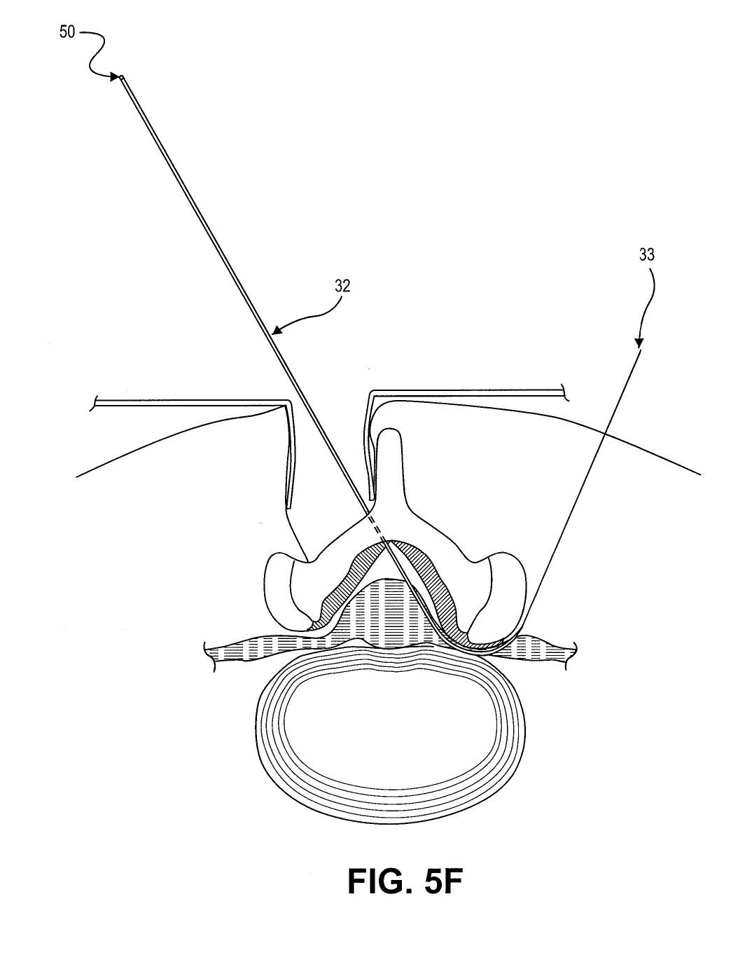

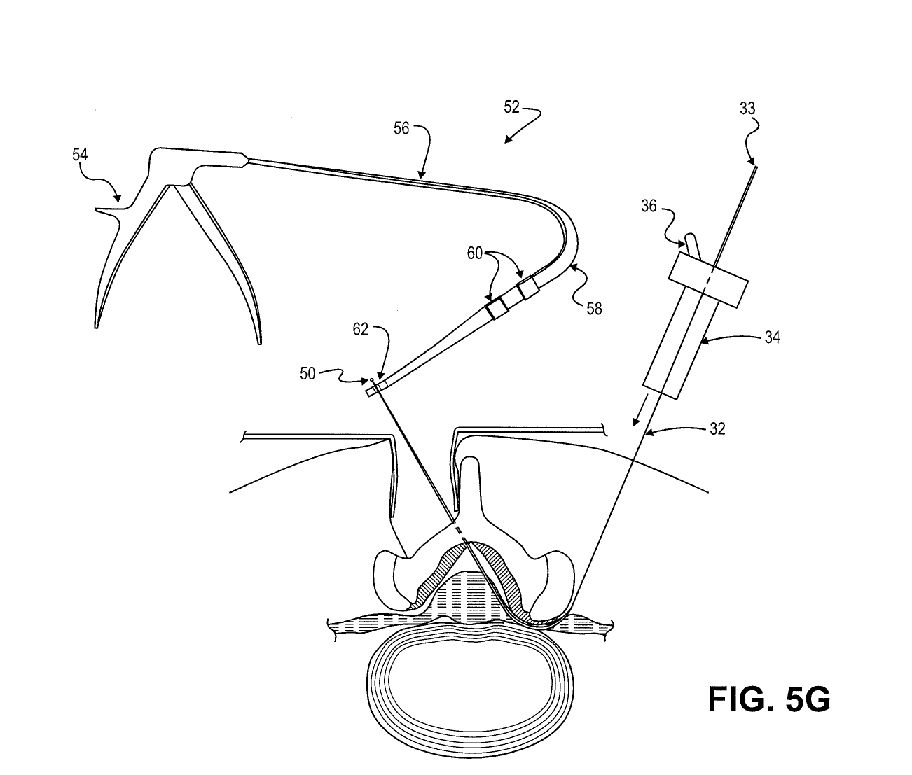

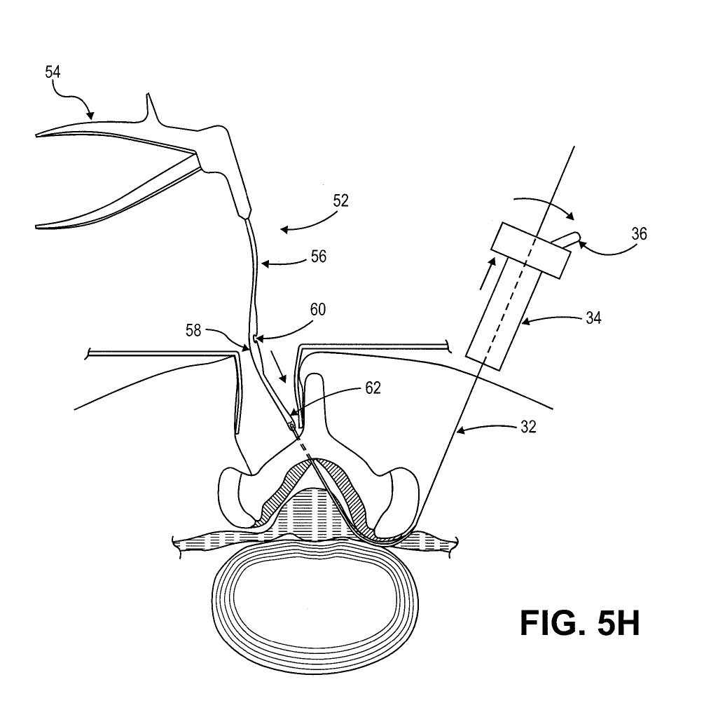

With reference now to FIGS. 5A-5I, one embodiment of a method for advancing a tissue modifying device into a patient's body using a guidewire delivery system is shown. Although this method is shown in reference to placement of a device in a spine, in various alternative embodiments, such a method may be used to place similar or alternative tissue modification devices in other locations in a human body, such as between tissues in a joint space, in the abdominal cavity, or in the carpal tunnel of the wrist, between bone and soft tissue in other parts of the body, and the like.

Referring to FIG. 5A, in one embodiment of a method for advancing a tissue modifying device, a probe 40 may be inserted into a patient's back using an open technique facilitated by retractors 42. Target tissues of a procedure, in this embodiment, may include ligamentum flavum (LF) and/or facet joint (F) tissue of a vertebra (V), which may impinge on non-target tissues, such as nerve root (NR) and/or cauda equina (CE), of the lumbar spine. Also depicted in FIG. 5A is an intervertebral disc (D). In FIG. 5B, a curved distal portion of probe 40 has been advanced to a position between target ligamentum flavum (LF) and non-target nerve root (NR) tissues. As depicted in FIG. 5C, in some embodiments, a curved guide member 46 may next be advanced out of an aperture on the curved distal portion of probe 40. In one embodiment, for example, guide member 46 may be housed within probe and advanced out of the distal aperture by advancing a slide member 44 on the shaft of probe 40. Next, as shown in FIG. 5D, guidewire 32 may be advanced through guide member 46 and out of the patient's back, using sharpened tip 33 to facilitate passage through the patient's back tissue. Probe 40 may then be removed, as shown in FIG. 5E, leaving guidewire 32 in place between the target and non-target tissues, as shown in FIG. 5F. Also shown in FIG. 5F is a shaped member 50 (in this embodiment, a ball) on the proximal end of guidewire 32.

Further description of methods, devices and systems for advancing a guidewire between tissues using a probe are provided in U.S. patent application Ser. No. 11/429,377, entitled “Spinal Access and Neural Localization” (Attorney-Docket No. 026445-000724US) and filed on Jul. 13, 2006, the full disclosure of which is hereby incorporated by reference. As described in that reference, in some embodiments, the curved distal portion of probe 40, curved guide member 46, or both may include one, two or more electrodes to help locate nerve tissue before placing guidewire 32. Such neural localization helps ensure that guidewire 32 is positioned between target and non-target tissue, which in turn helps ensure that a tissue modification device (or devices) placed using guidewire 32 are oriented so that a tissue modifying portion (or portions) of the device face and act on target tissue and not on non-target tissue such as neural tissue.

Referring now to FIG. 5G, once guidewire 32 is positioned between tissues, its proximal end with shaped member 50 may be coupled with a coupling member 62 on a distal end of a tissue modification device 52. Tissue modification device 52, in one embodiment, may include a proximal handle 54, a rigid proximal shaft portion 56, a flexible distal shaft portion 58, tissue cutting blades 60, and coupling member 62. Coupling member 62, various embodiments of which are described in greater detail below, may be either attached to or formed in distal shaft portion 58. In some embodiments, such as the one depicted in FIG. 5G, to attach guidewire 32 to coupling member 62, guidewire 32 may be laid into a channel on coupling member 62, and guidewire 32 and/or distal portion 58 may be rotated, relative to one another, to lock shaped member 50 into coupling member. Various alternative embodiments for coupling guidewires 32 with coupling members 62 are described in greater detail below. Before, after or during coupling of guidewire 32 and tissue modification device 52, guidewire 32 may also be coupled with distal guidewire handle 34, such as by advancing distal handle 34 over guidewire 32 (solid-tipped arrow).

As depicted in FIG. 5H, tightening member 36 may next be moved (curved, solid-tipped arrow) to tighten distal handle 34 around guidewire 32. Distal handle 34 may then be pulled (straight, solid-tipped arrow) to pull guidewire 32 and thus advance distal shaft portion 58 of tissue modification device 52 into place between target and non-target tissues in the spine, as shown in FIG. 51. Once device 52 is positioned as desired, as depicted in FIG. 51, proximal handle 54 and distal handle 34 may be pulled (straight, solid-tipped arrows), to apply tensioning force to guidewire 32 and device 52 and thus urge flexible portion 58 and blades 60 against target tissue, such as ligamentum flavum (LF) and/or facet joint (F) tissue. Handle 54 may then be actuated (curved, double-tipped arrow) to cause blades 60 to cut target tissue. When a desired amount of tissue is cut, guidewire 32 may be released from distal handle 34, and tissue modification device 52 and guidewire 32 may be removed from the patient's back. This method for advancing tissue modification device 52 using guidewire 32 is but one exemplary embodiment.

Various aspects of the method embodiment just described, such as the number or order of steps, may be changed without departing from the scope of the invention. Furthermore, a number of alternative embodiments of various devices and device elements are described below, which may be used in various embodiments of such a method. For example, in one alternative embodiment (not shown), probe 40 and tissue modification device 52 may be combined into one device. Such a device may include a guidewire lumen through which guidewire 32 may be passed. The combined device may be partially inserted into a patient, and guidewire 32 advanced between target and non-target tissues through the guidewire lumen. Shaped member 50 of guidewire 32 may then catch on one or more coupling members 62 of the combined device, to allow the device to be pulled into position between the target and non-target tissues. Guidewire 32 may then further be used to help apply tensioning force to the device to urge an active portion against target tissues. In another alternative embodiment, access to the intervertebral foramen may be achieved using a lateral approach, rather than a medial approach. These are but two examples of many alternative embodiments, and a number of other alternatives are contemplated.

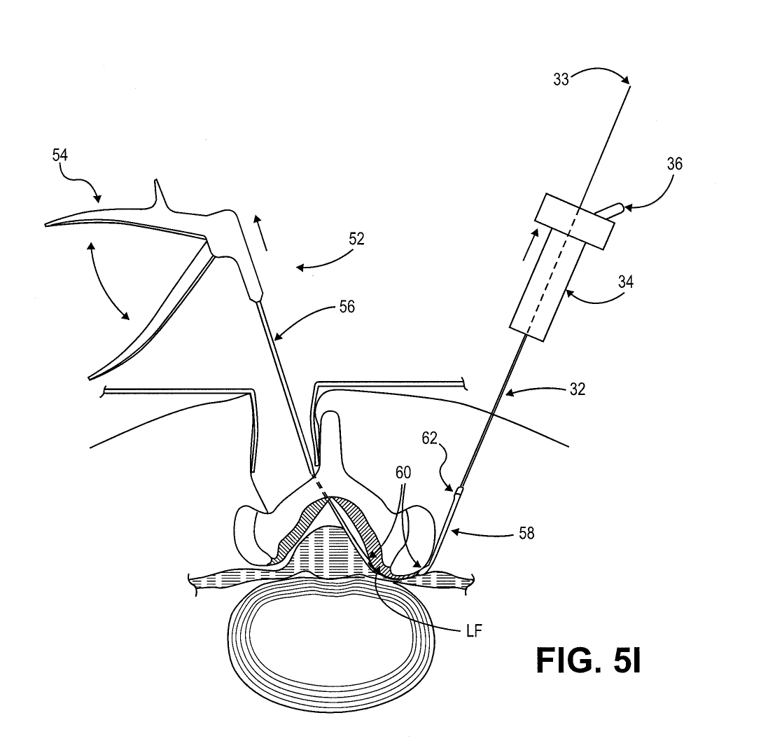

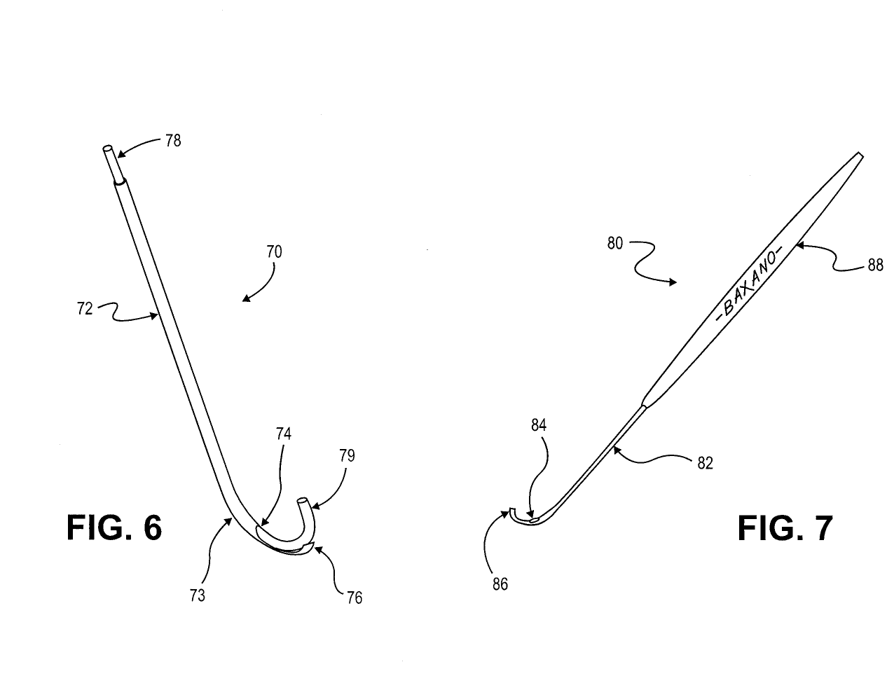

With reference now to FIG. 6, In one embodiment a probe 70 may include a thin, rigid tube 72 having one or more bends 73, a lumen (not visible) and a distal aperture 74, through which a flexible, curved tubular member 78 may be passed. Probe 70 may also include an angled distal tip 76 (or “toe”), which may be formed in a flattened distal portion of tube 72 and which may help guide a curved portion 79 of tubular member 78 in a desired direction. Tubular member 78 generally includes curved portion 79 and a lumen with sufficient inner diameter to allow a guidewire to pass therethrough.

In one embodiment, tube 72 is made from a hypotube-a narrow, thin, metallic tube generally used for medical applications. In alternative embodiments, tube 72 may be made from any of a number of suitable materials, such as but not limited to metals, polymers, ceramics, or composites thereof. Suitable metals may include, but are not limited to, stainless steel (303, 304, 316, 316L), nickel-titanium alloy, tungsten carbide alloy, or cobalt-chromium alloy, for example, Elgiloy® (Elgin Specialty Metals, Elgin, Ill., USA), Conichrome® (Carpenter Technology, Reading, Pa., USA), or Phynox® (Imphy SA, Paris, France). Suitable polymers include, but are not limited to, nylon, polyester, Dacron®, polyethylene, acetal, Delrin® (DuPont, Wilmington, Del. ), polycarbonate, nylon, polyetheretherketone (PEEK), and polyetherketoneketone (PEKK). Ceramics may include, but are not limited to, aluminas, zirconias, and carbides. Tubular member 78 is generally made of a flexible material, such as one of the polymers listed above.

Referring now to FIG. 7, in another embodiment, a probe 80 includes a handle 88, a tube 82 having a distal aperture 84 and an angled distal tip 86. As in the previous embodiment, a flexible tubular member (not shown) may be passed through tube 82 and distal aperture 84 to facilitate placement of a guidewire. Tube 82 may continue into and through at least part of handle 88 and may end proximally in a proximal aperture (not visible) at or near a proximal end of handle 88. Handle 88 may be made of metal, polymer, ceramic or other suitable material(s), and may be attached to tube 82 by any suitable technique. For example, in one embodiment, handle 88 may comprise a polymeric material injection molded over tube 82. In another embodiment, handle 88 may comprise a metallic material welded to tube 82.

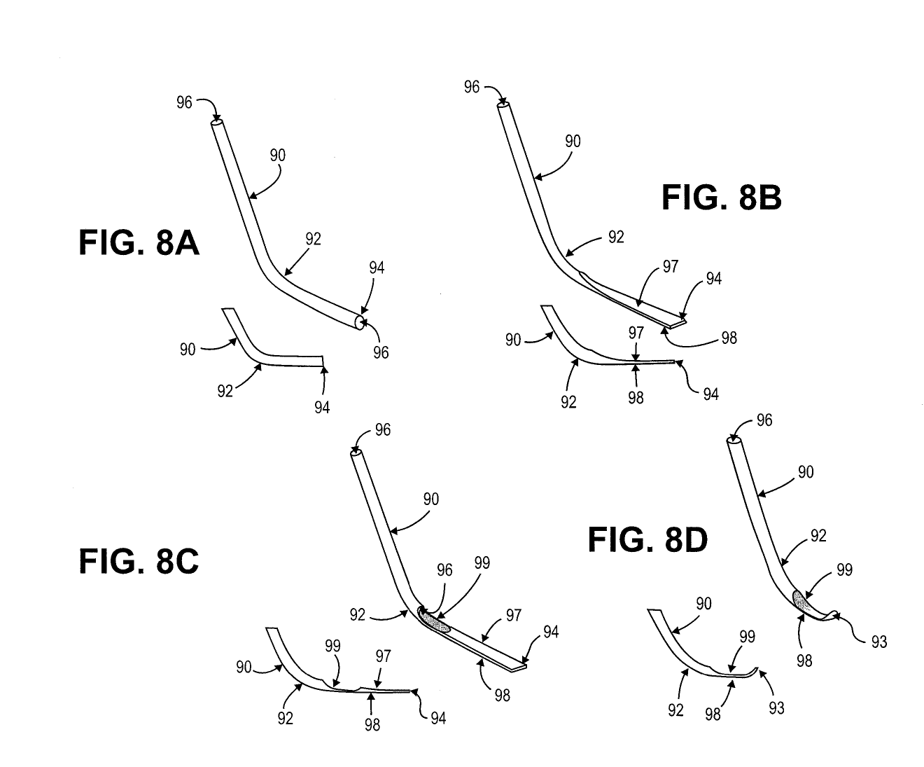

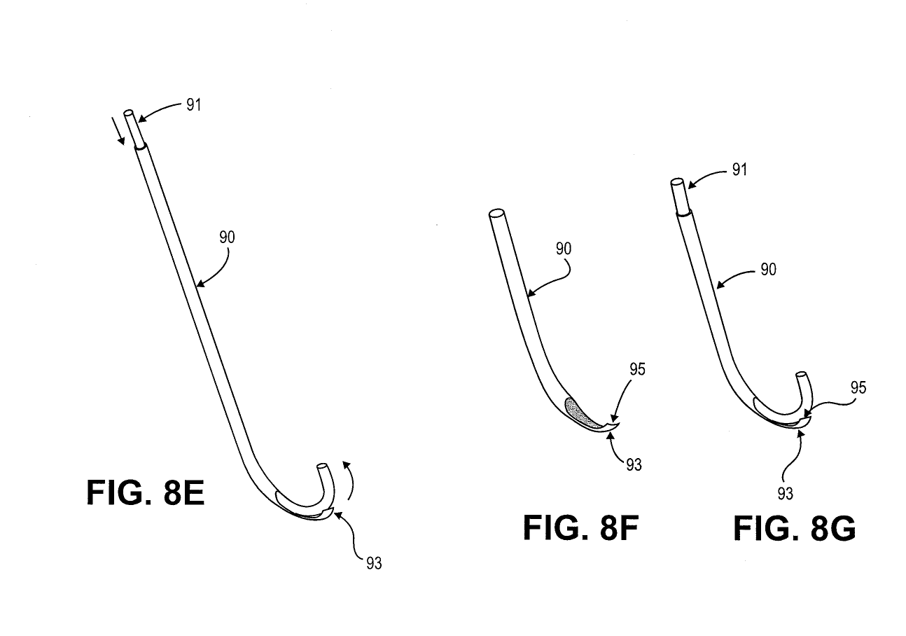

Referring now to FIGS. 8A-8G, one embodiment of a method for making a probe device is demonstrated. (In FIGS. 8A-8D and 8F-8G, only a distal portion of a probe device is shown. In FIGS. 8A-8D, the upper/right version of each figure is in perspective view, and the lower/left version is in side view.) As shown in FIG. 8A, in one embodiment, a bend 92 may be formed in a hypotube 90 (or other thin, rigid tube) having a lumen 96 and a distal end 94. Referring to FIG. 8B, before or after bend 92 is formed, a distal portion of hypotube 90 may be flattened to form an upper surface 97 (facing the acute angle of bend 92) and a lower surface 98 (facing the oblique angle of bend). As in FIG. 8C, a portion of upper surface 97 may then be removed to form an aperture 99 into lumen 96. Optionally, a portion of upper surface 97 and lower surface 98 distal to the removed portion may be removed, as in FIG. 8D, so that there is no longer any upper surface. In other words, and as shown in FIG. 8D, aperture 99 opens onto lower surface 98 of the flattened distal portion. In another optional step, the extreme distal end of the remaining lower surface 98 may be turned up (or “angled”) to form an angled distal tip 93 (or “toe”).

With reference now to FIG. 8E, in another optional step, a flexible, curved, tubular member 91 may be inserted (hollow-tipped arrows) into tube 90. A curved portion of tubular member 91 may extend out of tube 90 distally and may be guided by angled distal tip 93. In some embodiments, an additional fabrication step may be employed, as shown in FIGS. 8F and 8G. In this step, a groove 95 may be formed in angled distal tip 93. As shown in FIG. 8G, groove 95 may help guide tubular member 91. For example, groove 95 may help prevent tubular member 91 from slipping off the side of lower surface 98.

In various alternative embodiments of the method just described, any of a number of changes may be made without departing from the scope of the invention. For example, the order of various method steps may be changed, some steps may be skipped or combined, or the like.

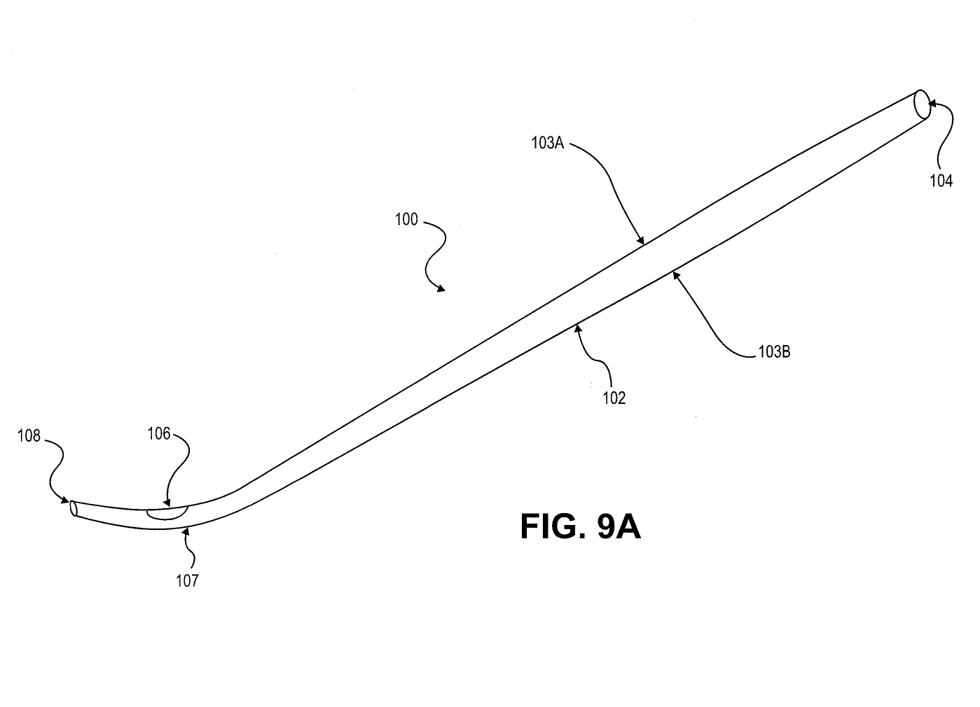

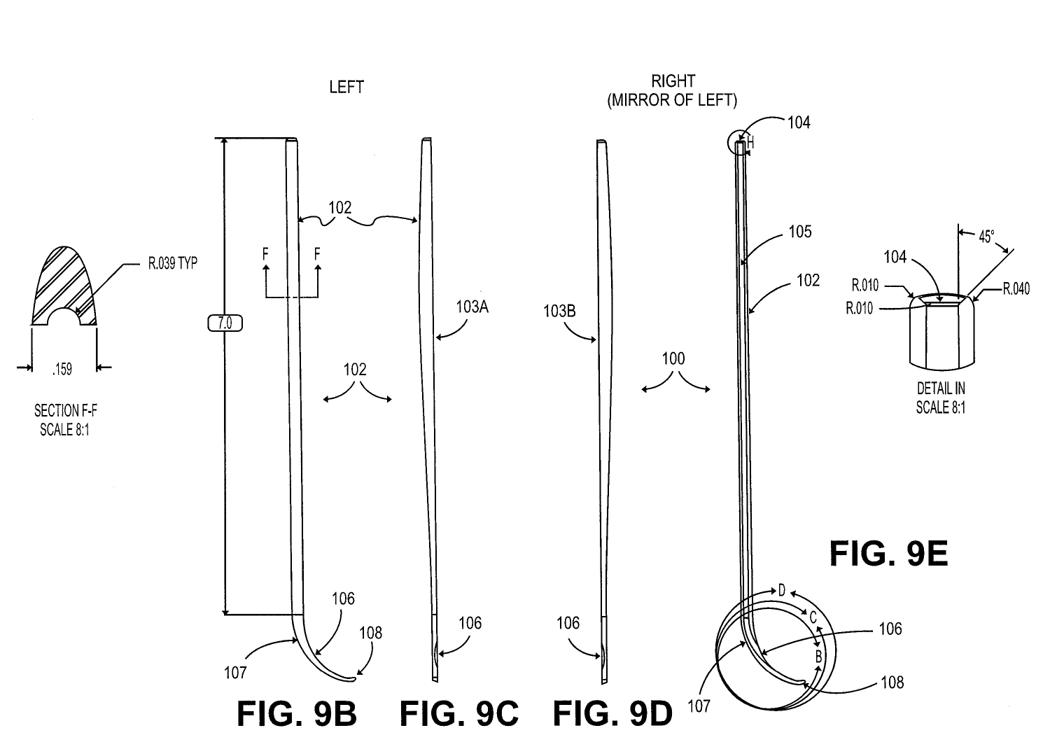

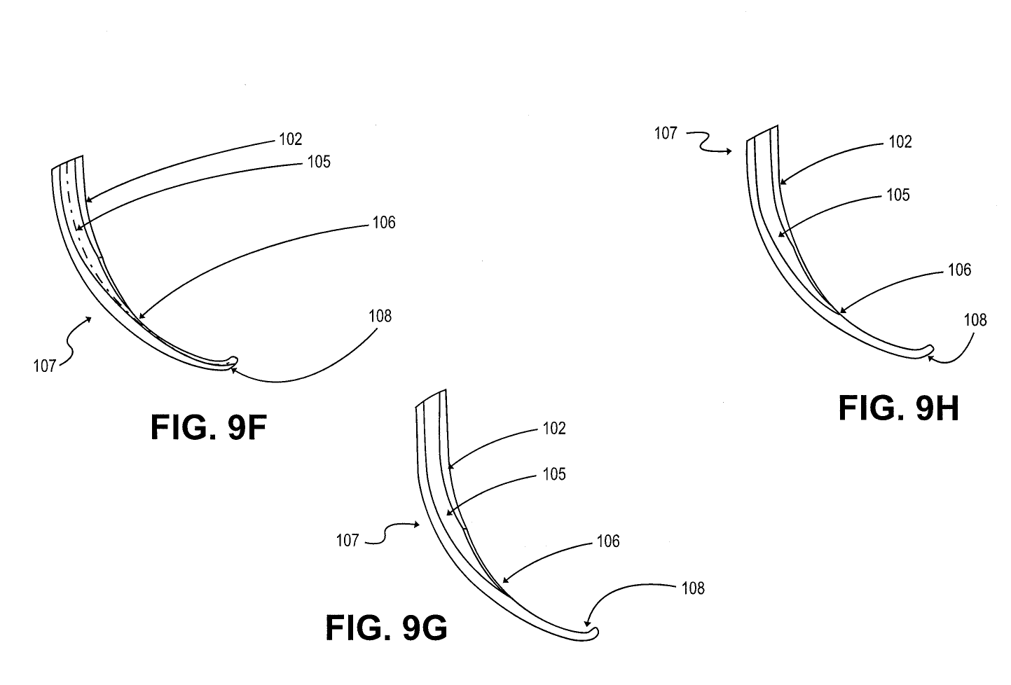

FIGS. 9A-9H show another embodiment of a probe device 100. As seen in FIG. 9A, in this embodiment, probe 100 includes a shaft 102 comprising two attached halves 103a, 103b and a curved distal portion 107. Shaft 102 also forms a lumen having a proximal aperture 104 and a distal aperture 106, and further includes an angled distal tip 108. Probe 100 may be made of stainless steel or any of an number of other suitable materials, such as those listed above.

FIG. 9B is a side view of probe 100. FIG. 9C is a frontal view of the left half 103a of shaft 102, and FIG. 9D is a frontal view of the right half 103b. FIG. 9E is a side, cross-sectional view, showing a lumen 105 of shaft 102. FIGS. 9F-9H are side, cross-sectional views of distal portion 107. The various shapes, sizes, angle measurements and the like shown in FIGS. 9A-9H describe one preferred embodiment of probe 100. It has been found that these shapes, sizes and angles may be preferable for advancing at least distal portion 107 into an epidural space of a spine and at least partway into an intervertebral foramen, using a contralateral approach. By contralateral approach, it is meant that probe 100 is advanced into the epidural space on one side of the spinous processes of the vertebrae and distal portion 107 is advanced into an intervertebral foramen on the opposite side. In an ipsilateral approach, probe 100 is advanced into the epidural space on one side and into a foramen on the same side.

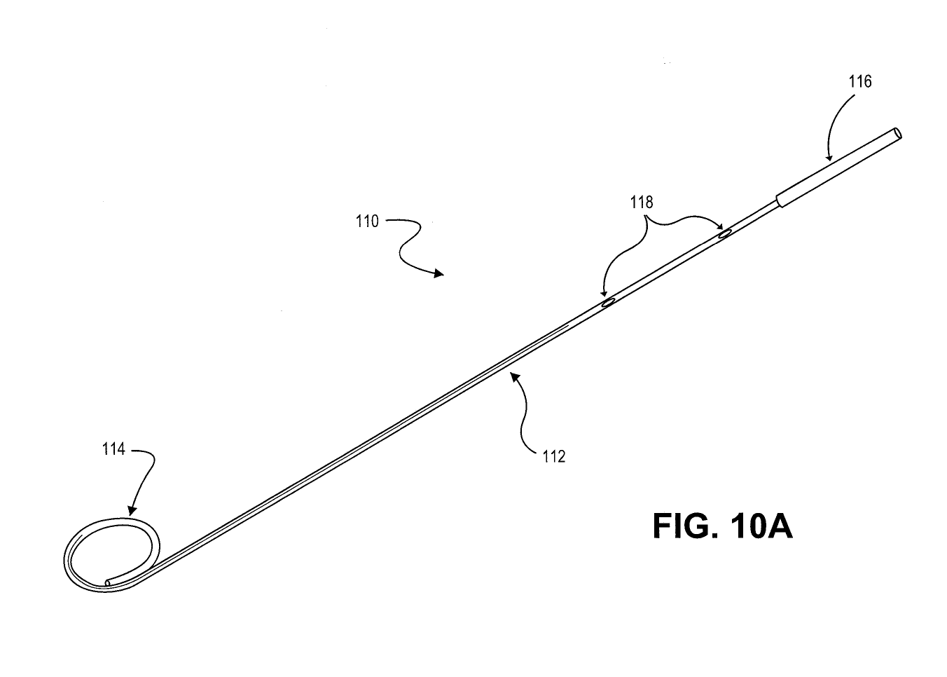

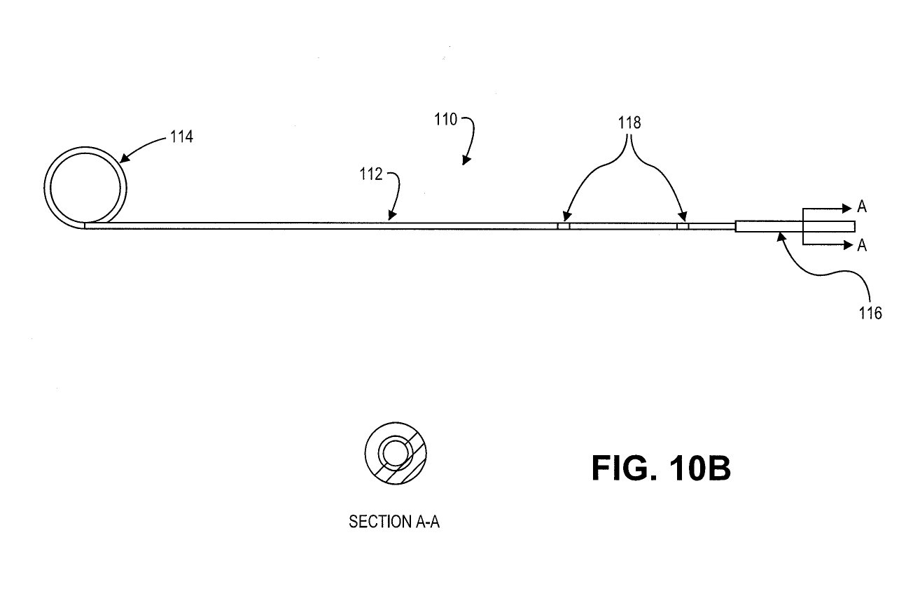

FIGS. 10A and 10B are perspective and side views, respectively, of a curved, flexible, tubular guide member 110 which may be used as part of a probe. Tubular member 110 may include a straight portion 112, a curved portion 114, markers 118 to indicate how far into a probe tubular member 110 has been inserted, and a stop 116 for coupling with the probe to prevent further insertion and/or to facilitate insertion and withdrawal of tubular member 110 from the probe. Tubular member 110 may be made of any suitable material, such as a flexible polymer, and generally has a lumen of sufficient diameter to allow passage of a guidewire. The various dimensions shown in FIGS. 10A and 10B are of one preferred embodiment of tubular member 110, and in alternative embodiments, tubular member 110 may have any of a number of other suitable configurations.

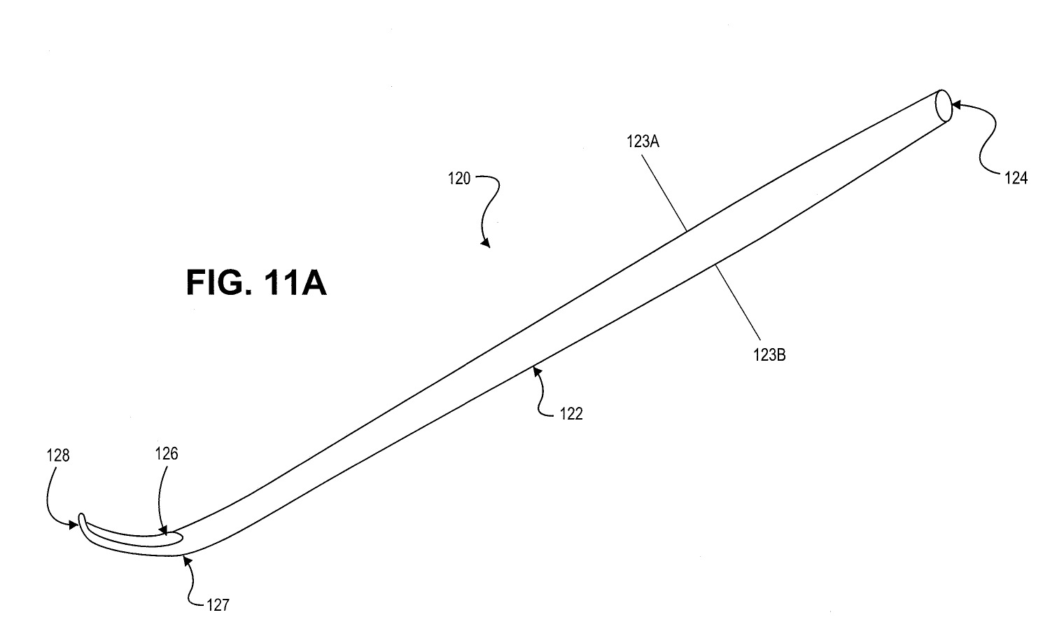

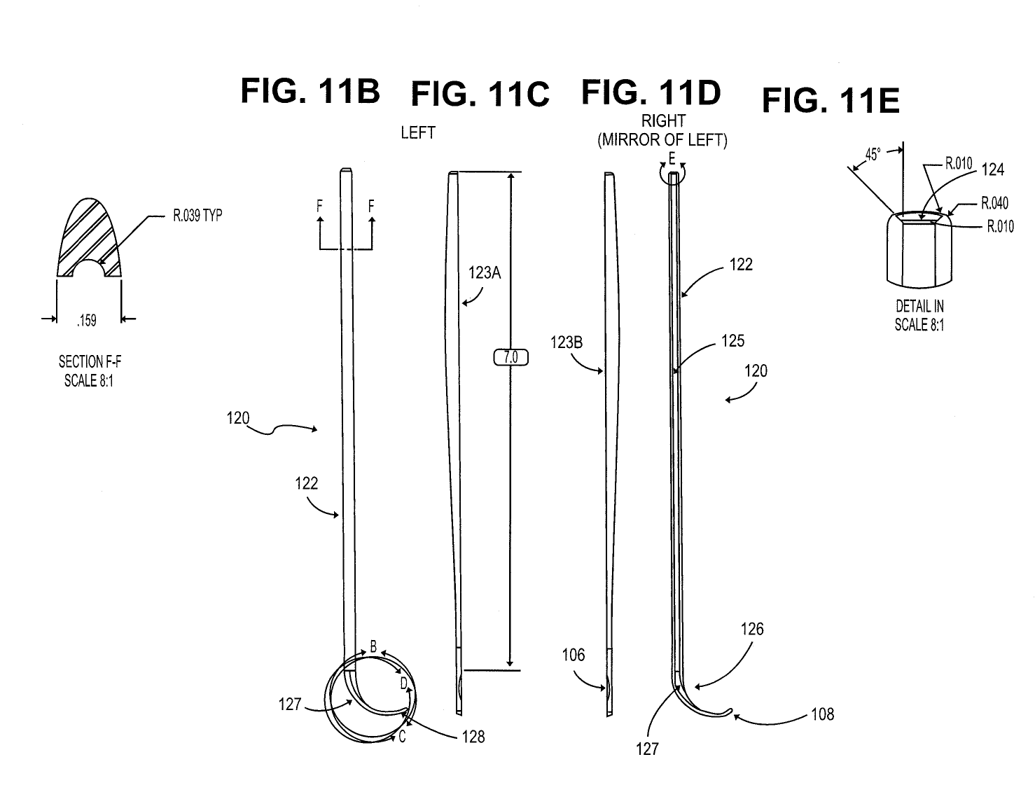

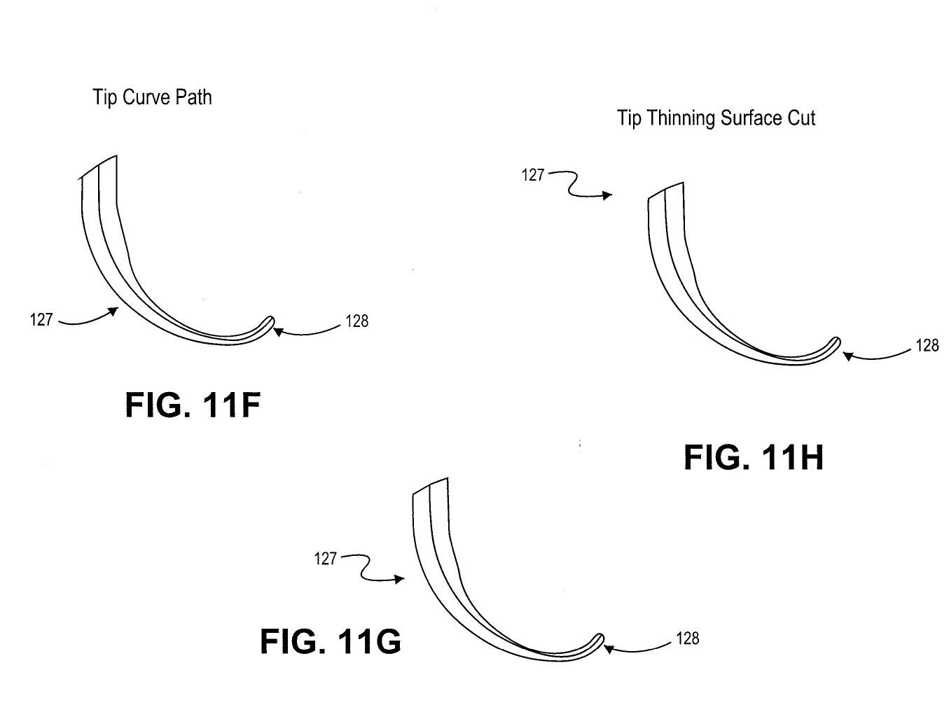

FIGS. 11A-11H show another embodiment of a probe device 120. As shown in FIG. 11A, in this embodiment, probe 120 includes a shaft 122 comprising two attached halves 123a, 123b and a curved distal portion 127. Shaft 122 also forms a lumen having a proximal aperture 124 and a distal aperture 126, and further includes an angled distal tip 128. Probe 120 may be made of stainless steel or any

FIG. 11B is a side view of probe 120. FIG. 11C is a frontal view of the left half 123a of shaft 122, and FIG. 11D is a frontal view of the right half 123b. FIG. 11E is a side, cross-sectional view, showing a lumen 125 of shaft 122. FIGS. 11 F-11H are side views of distal portion 127. The various shapes, sizes, angle measurements and the like shown in FIGS. 11A-11H describe one preferred embodiment of probe 120. It has been found that these shapes, sizes and angles may be preferable for advancing at least distal portion 127 into an epidural space of a spine and at least partway into an intervertebral foramen, using an ipsilateral approach.

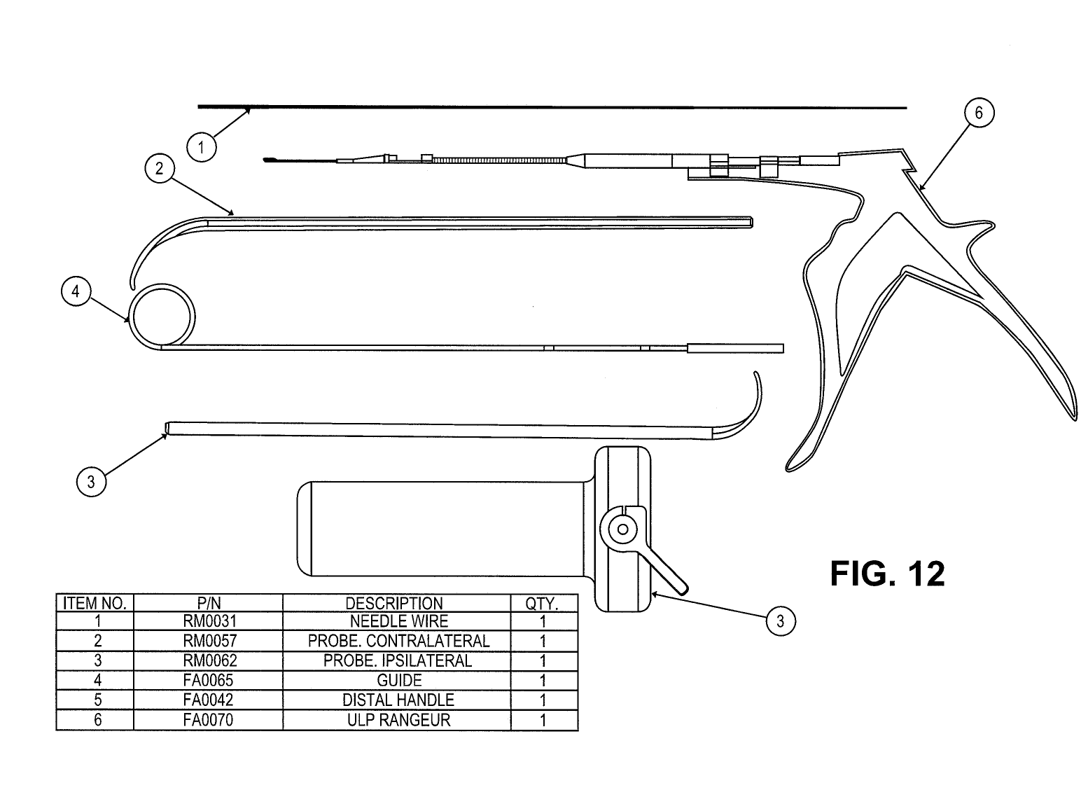

Referring to FIG. 12, one embodiment of a system for accessing and modifying tissue in a spine is shown. In this embodiment, the system may include a guidewire 1, a contralateral probe 2, an ipsilateral probe 3, a flexible, curved guide member 4, a distal guidewire handle 5, and a tissue modification device 6 (such as an ultra-low-profile rongeur, as shown). In alternative embodiments, the system may include any other combination of these or devices described herein or in the various patent applications incorporated by reference.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

We claim:

1. A method for making a probe for placing a guidewire between two tissues in a patient's body, the method comprising:
forming at least one bend in a thin, rigid tube having a proximal end and a distal end, wherein the bend is formed closer to the distal end than the proximal end;
flattening at least part of the tube closer to the distal end than the proximal end, wherein the flattened part comprises an upper surface facing the acute angle of the bend and a lower surface facing the oblique angle of the bend; and
removing a portion of the upper surface of the flattened part of the tube to form an aperture.

2. A method as in claim 1, wherein the aperture extends onto the bend in the tube.

3. A method as in claim 1, further comprising bending the distal end of the tube to form an angled distal tip.

4. A method as in claim 3, further comprising cutting off a distal end of the tube to form a new distal end, wherein the removed portion of the upper surface extends to the new distal end.

5. A method as in claim 4, wherein bending the distal end to form the angled tip comprises bending the new distal end.

6. A method as in claim 5, further comprising forming a groove in the distal tip for guiding a curved, flexible, tubular member.

7. A method as in claim 1, further comprising passing a curved, flexible, tubular member through the rigid tube.

8. A method as in claim 7, wherein the curved tubular member includes a curved portion toward a distal end, and wherein the curved member comprises a shape memory material capable of transitioning from a straight configuration, while passing through a straight portion of the rigid tube, to a curved portion, upon passing through the aperture of the rigid tube.

9. A method as in claim 1, further comprising forming a handle over a proximal portion of the tube.

10. A method as in claim 1, wherein at least a distal portion of the tube is configured to be passed into an epidural space and into an intervertebral foramen of a spine.

11. A method for making a probe for placing a guidewire between two tissues in a patient's body, the method comprising:
forming two halves of a metal probe comprising a proximal handle portion and a curved distal portion; and
coupling the two halves of the probe together, such that a lumen is formed through the two halves, extending from the proximal handle through at least a portion of the curved distal portion.

12. A method as in claim 11, further comprising passing a curved, flexible, tubular member through the lumen of the probe.

13. A method as in claim 12, wherein the curved tubular member includes a curved portion toward a distal end, and wherein the curved member comprises a shape memory material capable of transitioning from a straight configuration, while passing through the handle of the probe, to a curved portion, upon passing out of the lumen.

14. A method as in claim 11, wherein at least the curved distal portion of the probe is configured to be passed into an epidural space and into an intervertebral foramen of a spine.

15. A probe for placing a guidewire between two tissues in a patient's body, the probe comprising:
a thin, rigid tube having a proximal straight portion, a flattened distal portion, a bend disposed between the proximal and distal portions, and a lumen passing through the proximal portion and at least part of the distal portion; and
a curved, flexible tubular member disposed at least partially within the lumen of the tube, wherein at least a portion of the tubular member is configured to change from a straight configuration in the proximal portion of the tube to a curved configuration upon exiting the tube.

16. A probe as in claim 15, wherein the distal portion of the tube straight.

17. A probe as in claim 15, wherein the distal portion of the tube is curved.

18. A probe as in claim 15, further comprising an angled distal tip at an extreme distal end of the distal portion of the tube, wherein the angled distal tip is angled relative to the distal portion.

19. A probe as in claim 15, wherein the lumen extends from a proximal aperture in the proximal portion of the tube to a distal aperture in the distal portion.

20. A probe as in claim 19, wherein the distal aperture extends from the bend to the distal portion.

21. A probe as in claim 15, wherein the flexible tubular member comprises a shape memory material.

22. A probe as in claim 15, wherein a lumen of the flexible tubular member has an inner diameter sufficient to allow passage of a guidewire therethrough.
